(19) United States
(12) Patent Application Publication
(10) Pub. No.: US 2004/0087972 A1
(43) Pub. Date: May 6, 2004

(54) Skin abrading apparatus

(76) Inventors: Robert Stephen Mulholland, Toronto (CA); Alan Broadwin, Larchmont, NY (US)

Correspondence Address: MASON & ASSOCIATES, PA, 17757 US HWY 19 N., SUITE 500, CLEARWATER, FL, 33764, US

(21) Appl. No.: 10/244,881

(22) Filed: Sep. 16, 2002

(51) Int. Cl. A61B 17/50

(52) U.S. Cl. 606/131

(57) ABSTRACT

A hand held skin abrading apparatus is provided. The apparatus comprises a handle configured for gripping by a human operator; a reciprocating motion generator, in the form of a magnetostrictive ultrasonic transducer, associated with the handle; an applicator attached to the handle and having an applicator blade for applying abrading motion to a surface and a coupler for coupling the applicator to the magnetostrictive ultrasonic transducer. The applicator is releasably securable to the handle of a skin abrading apparatus and includes a connector portion extending from the applicator blade, the connector portion having a central axis, wherein the applicator blade is offset from the central axis to enable the apparatus to be operated with the handle above the plane of the surface being abraded. The apparatus may include provision for irrigation of the surface being abraded.

### FIELD OF THE INVENTION:

The invention relates to a skin-abrading device, generally. In particular, the invention relates to hand held skin abrading devices.

### BACKGROUND OF THE INVENTION

People with skin imperfections, such as fine lines and wrinkles, pigment abnormalities and acne or sun damaged skin, have long sought to have them removed. Over the past 100 years, several methods and devices have been employed to this end. For example, chemical peels, dermabrasive devices (such as high speed burrs, abrasive paper or wire brushes), lasers and ultrasonic devices have been used to remove layers of the stratum corneum of the epidermis. Each of these methods and devices, however, has important shortcomings that preclude their widespread use.

Dermabrasive devices, such as wire brushes, fraises and abrasive papers usually require a topical anesthetic and result in a “brush burned” feeling, some swelling and newly formed pink skin. Healing takes place usually over a 7 to 10 day period, with a return to normal appearance usually in 8 to 12 weeks. Further, controlled removal of epidermal layers is very difficult and requires considerable skill. Chemical peels require the use of glycolic or other acids, and carry the difficulties and risks associated with the application of an active liquid to the face, especially near the eyes. Lasers are a very expensive alternative, which requires significant training and special precautions associated with laser safety. Further, it is not easy to cover broad areas of the face evenly and quickly.

Although ultrasonic vibrating members utilizing piezoelectric transducers have demonstrated the ability to remove tissue delicately and precisely, layer by layer. These devices have in the past been prone to fatigue failure and have not been able to sustain a desired motion under load.

It is an object of the present invention to provide an ultrasonic skin abrading device that can remove tissue delicately and precisely, is not prone to fatigue failure and is able to sustain its intended motion under load.

### SUMMARY OF THE INVENTION

The present invention provides an ultrasonic skin abrading apparatus comprising a handle configured for gripping by a human operator; a reciprocating motion generator, in the form of a magnetostrictive ultrasonic transducer, associated with the handle; and a coupler for coupling the magnetostrictive ultrasonic transducer to an applicator. The applicator includes an applicator blade for applying abrading motion to a surface.

The invention further provides that the magnetostrictive ultrasonic transducer may comprise a stack of magnetostrictive laminations insertable into the cavity of the handle, which includes at least one electronically conducting coil for surrounding the stack and inducing an electromagnetic field in the stack in response to electrical current flow in the coil thereby causing longitudinal movement in the stack.

The present invention further provides an applicator for use with a skin abrading apparatus, wherein the applicator is releasably securable to the handle of a skin abrading apparatus and includes an applicator blade for applying a reciprocating abrading motion.

The invention further provides that the applicator may include a connector portion extending from the applicator blade, the connector portion having a central axis, wherein the applicator blade is offset from the central axis to enable the apparatus to be operated with the handle above the plane of the surface being abraded.

The applicator blade may include at least one hole extending therethrough for allowing fluid passage therethrough onto an underlying surface.

### BRIEF DESCRIPTION OF THE DRAWINGS

Preferred embodiments of the present invention are described below with reference to the accompanying drawings in which:

FIG. 1 is a schematic view illustrating a skin abrading apparatus according to the present invention connected to an ultrasonic converter/generator;

FIG. 2 is an axial section of a handle of a skin abrading apparatus according to the present invention illustrating schematically the parts housed within;

FIG. 3 is a schematic view illustrating a magnetostrictive ultrasonic transducer component of a skin abrading apparatus according to the present invention and an attached coupler;

FIGS. 4(a-b) are axial sectional views illustrating alternate embodiments of a coupler component of a skin abrading apparatus according to the present invention;

FIGS. 5(a-b) are axial sectional views illustrating alternate embodiments of an applicator component for a skin abrading apparatus according to the present invention and an attached coupler.

FIG. 6a is a side elevation sectional view of an applicator component for a skin abrading apparatus according to the present invention.

FIG. 6b is a plan view of the applicator component of FIG. 6a; and,

FIGS. 7(a-i) are side elevations illustrating alternate embodiments of an applicator blade component of an applicator for a skin abrading apparatus according to the present invention.

### DETAILED DESCRIPTION

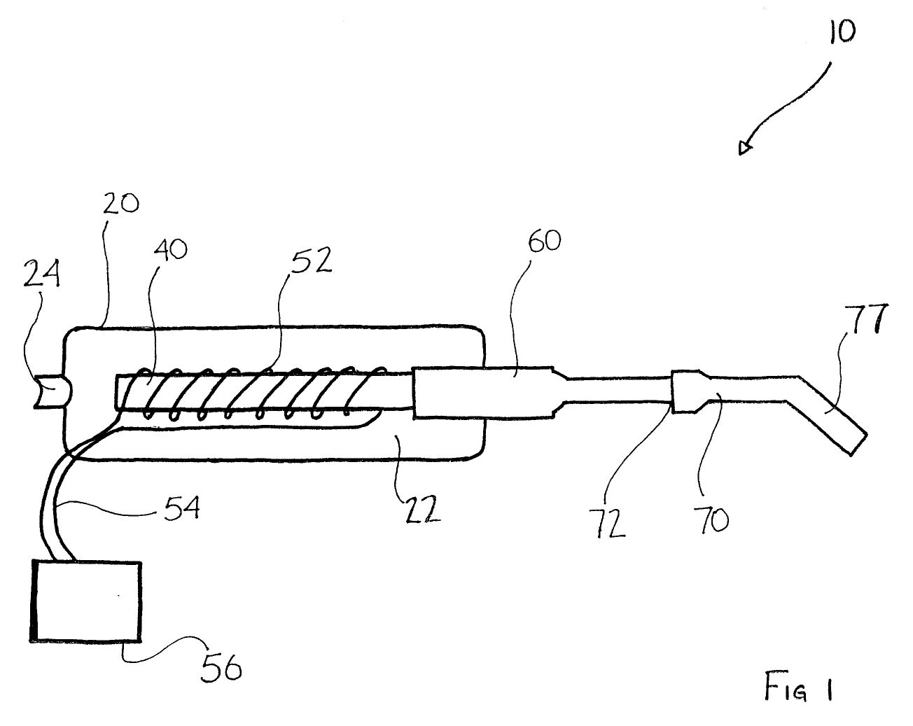

FIG. 1 illustrates a skin abrading apparatus (SAA) 10 made in accordance with a preferred embodiment of the invention. The SAA 10 is adapted to apply abrading motion to a surface by using reciprocating motion generated by a magnetostrictive ultrasonic transducer (MUT) 40.

The SAA 10 includes a handle 20 configured for gripping by a human operator; a reciprocating motion generator, in the form of the MUT 40 associated with the handle 20; a coupler 60 for coupling the MUT 40 to an applicator 70. The applicator 70 has an applicator blade 77 for applying abrading motion to a surface.

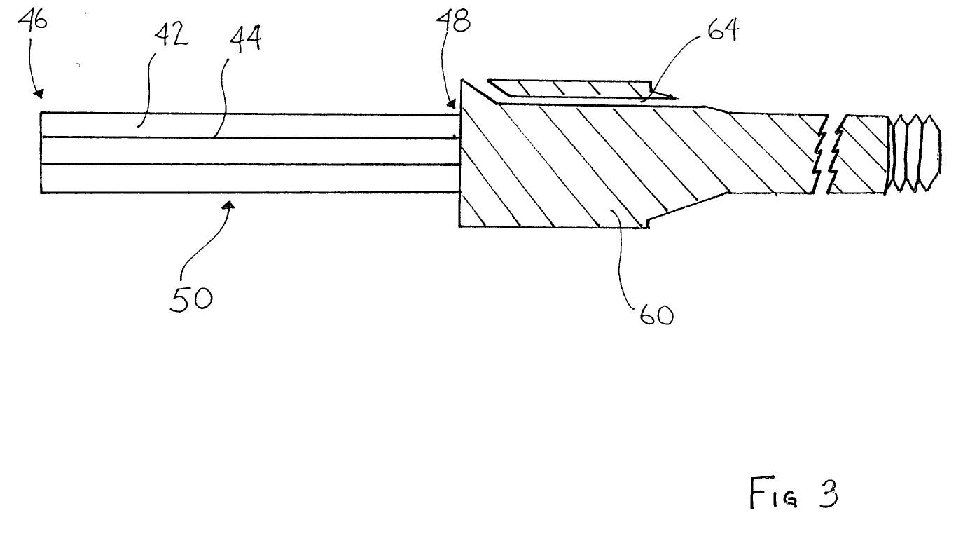

Referring now to FIGS. 1-3, the handle 20 includes a cavity 22 that is configured to receive and extend about the MUT 40. A fluid inlet 24 fluidly communicates with the cavity 22 for supplying fluid to the cavity 22 from a fluid source (not shown). A fluid outlet 64, fluidly communicates with the cavity 22 for discharging fluid from the cavity 22. Since the reciprocating motion of the MUT 40 generates heat, a cooling fluid is circulated through the cavity 22 in order to prevent the MUT 40 and handle 20 (and the components it houses) from overheating. In a preferred embodiment of the invention, water is used as the cooling fluid. In an alternate embodiment, a saline solution is used. However, any other fluid known in the art, which acts to cool the heated handle components may be used. To avoid leakage, an O-ring 28 is positioned between the inner surface 30 of the handle 20 and an outer surface 62 of coupler 60, thereby creating a leak resistant seal.

Fluid may for be provided from a sterile fluid bag pressurized with an inflatable cuff, such as a blood-pressure cuff. Other sources of pressurized fluid will no doubt be apparent to those skilled in the relevant art. According to a preferred embodiment of the present invention, the cooling fluid may also be used to irrigate the area being abraded. In such an arrangement, the selected fluid must be compatible with the human body.

The MUT 40 comprises a stack 50 of magnetostrictive laminations 42 that are fabricated from a magnetostrictive material, such as a nickel alloy. The laminations 42 are coated with a layer 44 of an oxide, which serves to insulate the adjacent laminations 42 from each other. The laminations 42 are brazed together at opposite ends 46 and 48, thereby forming the magnetostrictive stack 50. The brazing of the ends 46 and 48 ensures that the laminations 42 remain bonded together during operation and also provides mechanical contact between the laminations 42 and the coupler 60. The applicator 70, coupler 60 and MUT 40 may be releasably securable to the handle 20 thereby permitting them to be separated from handle 20 for maintenance and to be sterilized.

The cavity 22 also includes at least one electronically conducting coil 52 for surrounding the stack 50 and inducing an electromagnetic field in the stack 50 in response to electrical current flow in the coil 52, thereby causing longitudinal movement in said stack. The coil 52 is connected to an ultrasonic generator 56 via connecting wires 54. The ultrasonic generator 56 is capable of generating a varying electrical current having an ultrasonic frequency. The ultrasonic generator 56 may for example be a Cavitron® Select™ SPSTM Ultrasonic Scaler, as sold by DENTSPLY® Professional of York, Pa., USA. The ultrasonic generator 56 converts a 50-60 Hz AC input current into an ultrasonic frequency output current, thereby causing the MUT 40 to vibrate at ultrasonic frequencies of between 28 and 32 kHz. In a preferred embodiment, MUT 40 vibrates at a vibrating frequency of 30 kHz.

When the ultrasonic current passes through the coil 52, a magnetic field is induced, the direction of which is parallel to a longitudinal axis 34 of the laminations 42 of the magnetostrictive stack 50. In response to the generated magnetic field, the stack 50 strains and exhibits a corresponding change in length. It is this change in length (or reciprocating amplitude of vibration), which results from the magnetic coupling between the wire coils 52 and the magnetostrictive stack 50, that is passed on to the applicator 70 (and therefore applicator blade 77) through the coupler 60. The resulting reciprocating amplitude of vibration of the applicator blade 77 would typically be between 12 and 37 microns. Testing suggests that preferably the applicator blade 77 have a reciprocating amplitude of vibration of 25 microns, peak to peak.

Advantages of using a magnetostrictive ultrasonic transducer rather than a piezoelectric transducer are realized in longevity, performance and safety. The magnetostrictive ultrasonic transducer is more rugged and acoustically less sensitive to frequency and loading. Furthermore, the power supply is electrically isolated from the magnetostrictive stack as it is magnetically rather than electrically coupled thereto. In contrast, a piezoelectric device requires a very high voltage to be applied to the piezoelectric crystals, which in turn are directly mounted to the applicator assembly requiring electrical isolation and carrying the attendant risks of the electrical isolation breaking down.

The SAA 10 may have a fluid conduit means for directing a cooling fluid, such as water or a saline solution, onto applicator blade 77. In a preferred embodiment of the invention, the fluid that is used for cooling the handle is discharged onto the applicator blade 77. Accordingly, the fluid conduit means may include at least one irrigation passage 64 extending from the cavity 22 and through the coupler 60.

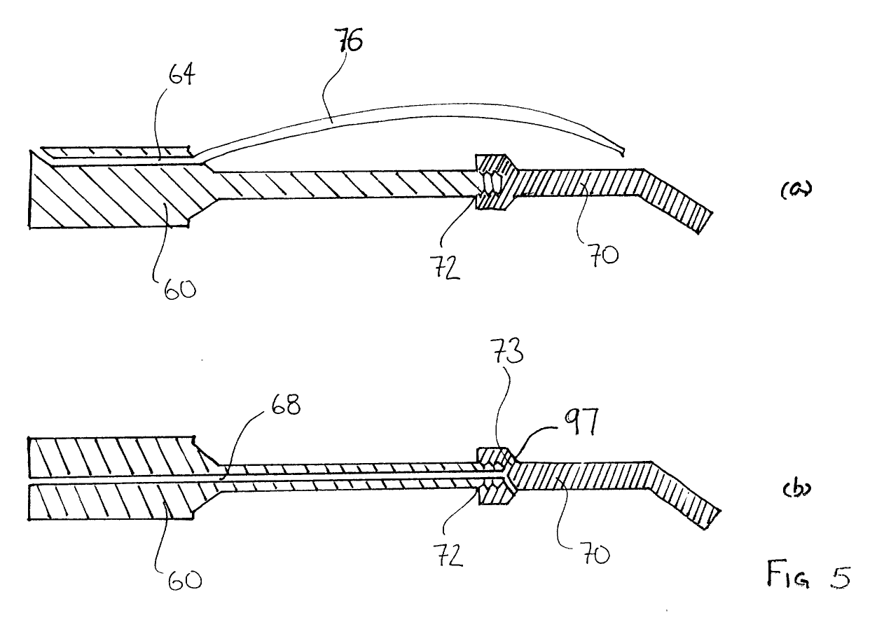

Referring now to FIGS. 1 and 5, the applicator 70 is releasably securable to the coupler 60. In a preferred embodiment of the invention, a connector portion 75 extends from the applicator 70 and attaches to the coupler 60 by a threaded joint 72, thereby enabling the applicator 70 to be removed for possible maintenance to replacement. However, any other attachment means known in the art, which permits the applicator 70 to be removed for possible maintenance to replacement, may be employed.

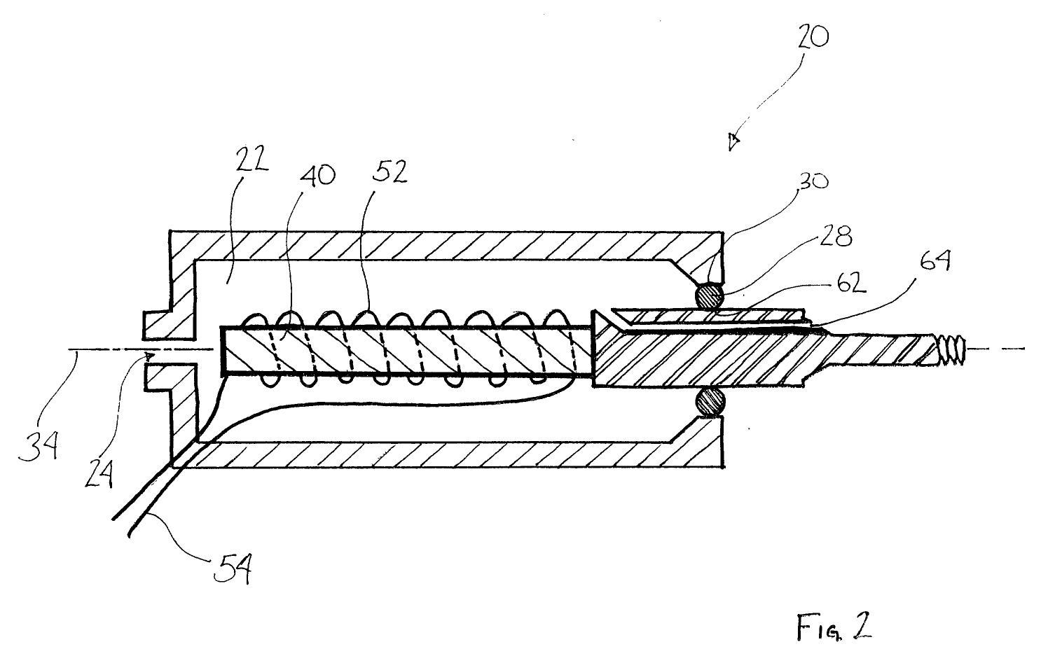

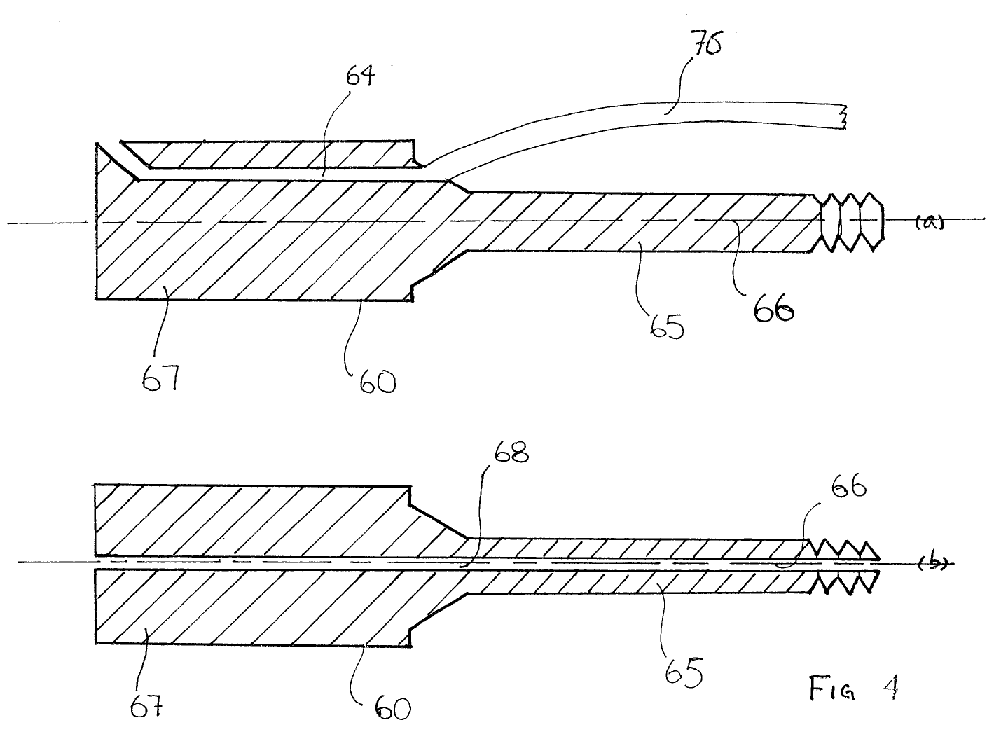

Referring now to FIGS. 2, 4 (a-b), 5 (a-b) and 6 (a-b), which are axial sectional views illustrating alternate embodiments of the coupler 60, the coupler 60 includes a shaft 65 extending to the applicator 70. The shaft 65 has a central longitudinal coupler axis 66. In a preferred embodiment illustrated in FIGS. 4a and 5a, the coupler 60 includes a fluid conduit means extending through the MUT connector portion 67 in the form of at least one irrigation passage 64 and a tube 76, which registers therewith and which combine to direct a cooling fluid such as water from the cavity 22 to the applicator blade 77, thereby cooling the abraded surface. In a preferred embodiment, the tube 76 is a hypodermic tube and extends to the applicator blade 77. However, any other tubing known in the art that can register with irrigation passage 64 and permit a cooling fluid to be deposited on to the applicator blade 77 may be employed. In an alternate embodiment illustrated in FIGS. 4b and 5b, the coupler 60 includes a fluid conduit means extending along the coupler axis 66 in the form of an irrigation passage 68, which registers with at least one irrigation passage 73 extending through the connector portion 75 of the applicator and terminating in an outlet 97. This enables a cooling fluid such as water or a saline solution, to flow from the cavity 22, through the fluid conduit means, out of the outlet hole 97 and on to the applicator blade 77, thereby cooling the abraded surface.

The applicator 70 is constructed preferably of 6AL4V Titanium because of its proven acoustic properties and durability. This material is also suitable for contact with human tissue and for operation in a saline environment without degradation. However, any other material known in the art, which has properties similar to those listed for 6AL4V Titanium, may be used. The grain direction of the titanium metal of the applicator 70 is preferably aligned with a longitudinal axis 90 of the applicator 70.

The length of the applicator 70 is prescribed by the acoustic design to be resonant at the selected frequency. The reciprocating motion of the MUT 40 will, at certain harmonic frequencies, generate a standing wave of vibration, which has nodes, corresponding to areas of minimum reciprocating amplitude of displacement, and anti-nodes, corresponding to areas of maximum reciprocating amplitude of displacement. In the preferred embodiment of the invention, the applicator 70 is of a length such that the applicator blade 77 is located at an anti-node.

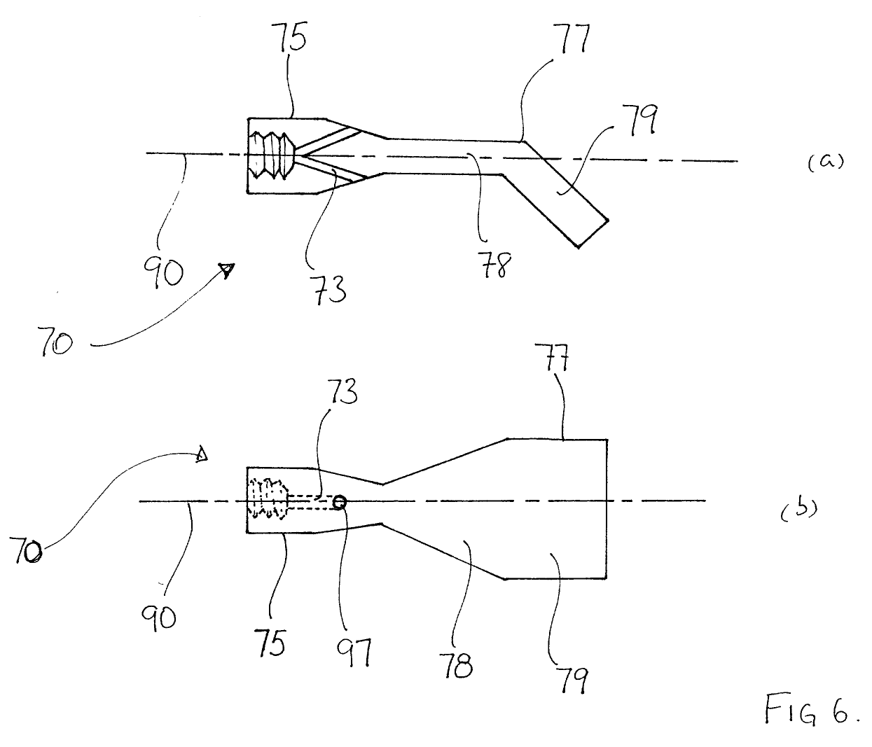

Referring now to FIGS. 6 (a-b) and 7 (a-i), which are side elevations illustrating alternate embodiments for the applicator blade 77, the applicator blade 77 is preferably spatulate in shape and includes a flared portion 78 and a rectangular portion 79.

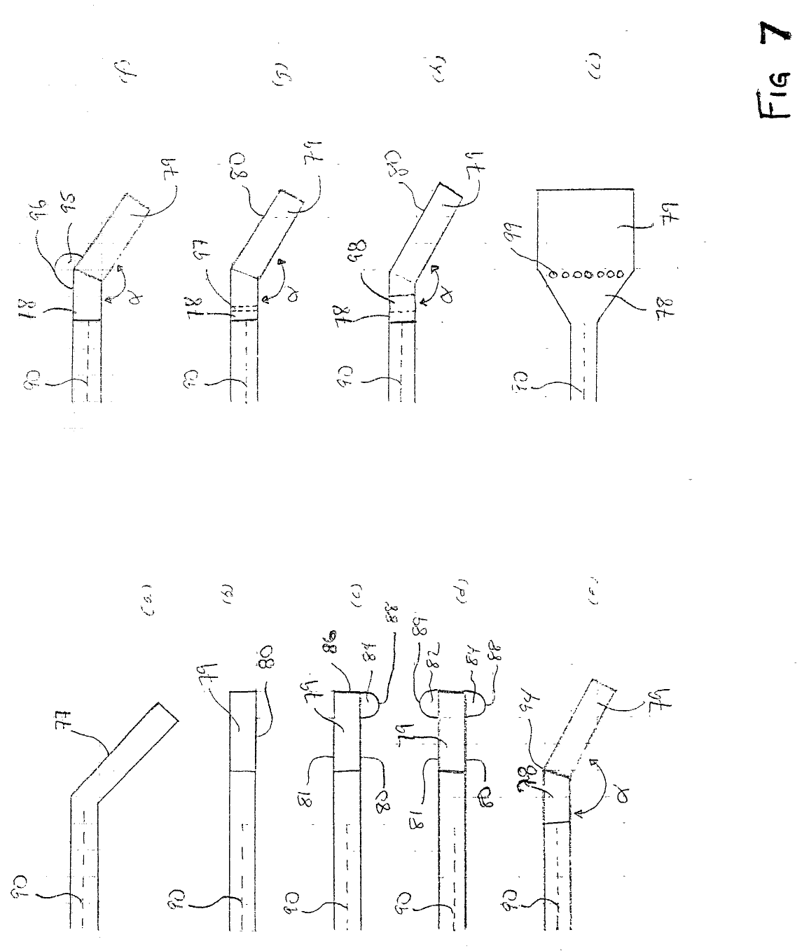

In the embodiment illustrated in FIG. 7a, the applicator blade 77 is offset from a central axis 90 of the coupler connector portion 75 to enable the SAA 10 to be operated with the handle 20 above the plane of the surface being abraded.

In the embodiment illustrated in FIGS. 7b-d, the applicator blade 77 is not offset from the central axis 90 and includes at least one abrading surface 80 and preferably a second abrading surface 81, as illustrated in FIGS. 7c-d. The abrading surface 80 may include at least one ridge 84 extending transversely across the abrading surface 80 and defining a ridge abrading surface 88. In the embodiment illustrated in FIG. 7c, the ridge 84 extends transversely across the abrading surface 80, adjacent to an outer edge 86 of the applicator blade 77. In the embodiment illustrated in FIG. 7d, a second ridge 82 extends transversely across the second abrading surface 81 opposite the ridge 84 and defines a second ridge abrading surface 89.

In the embodiment illustrated in FIGS. 7e-h, the rectangular portion 79 is offset from the central axis 90 to enable the SAA 10 to be operated with the handle 20 above the plane of the surface being abraded. The rectangular portion 79 meets the flared portion 78 at an angle α of between 90 degrees and 180 degrees relative to the central axis 90. In a preferred embodiment, the rectangular portion 79 meets the flared portion 78 at an angle α of 150 degrees.

In the embodiment illustrated in FIGS. 7e-f, the angle α defines an outer vertex 94, adjacent to which at least one ridge 95 extends and defines a ridge abrading surface 96. FIG. 7f illustrates a preferred embodiment, where the ridge 95 is located on the outer vertex 94.

In the embodiment illustrated in FIG. 7g, the applicator blade 77 includes at least one hole 97 extending therethrough for allowing a cooling fluid, such as water or a saline solution, passage therethrough onto the abrading surface 80. FIG. 7h illustrates an embodiment where the at least one hole is a slot 98. In the embodiment illustrated in FIG. 7i, a plurality of holes is located on the applicator blade 77.

The present invention is defined by the claims appended hereto, with the foregoing description being illustrative of the preferred embodiments of the invention. Those of ordinary skill may envisage certain additions, deletions and/or modifications to the described embodiments, which, although not explicitly suggested herein, do not depart from the scope of the invention, as defined by the appended claims.

We claim:

1. A skin abrading apparatus comprising:
a handle configured for gripping by a human operator;
a reciprocating motion generator associated with said handle;
an applicator attachable to said handle and having an applicator blade for applying abrading motion;
a coupler for coupling said applicator to said reciprocating motion generator and transferring reciprocating motion from said reciprocating motion generator to said applicator blade; wherein,
said reciprocating motion generator is a magnetostrictive ultrasonic transducer.

2. The apparatus of claim 1, wherein said handle includes a cavity, a fluid inlet fluidly communicating with said cavity for supplying fluid to said cavity from a fluid source, and a fluid outlet, fluidly communicating with said cavity for discharging fluid from said cavity.

3. The apparatus of claim 2, wherein said cavity receives and extends about said magnetostrictive ultrasonic transducer.

4. The apparatus of claim 3, wherein said magnetostrictive ultrasonic transducer further comprises a stack of magnetostrictive laminations insertable into said cavity and said cavity includes at least one electronically conducting coil for surrounding said stack and inducing an electromagnetic field in said stack in response to electrical current flow in said coil thereby causing longitudinal movement in said stack.

5. The apparatus of claim 4, wherein said coupler mechanically couples said applicator to said stack and said apparatus has a fluid conduit means associated with said fluid outlet for directing said fluid onto said applicator blade.

6. The apparatus of claim 5, wherein said at least one electronically conducting coil is connected to an ultrasonic generator capable of generating an electrical current having an ultrasonic frequency.

7. The apparatus of claim 6, wherein said ultrasonic generator converts a 50-60 Hz AC input current into an ultrasonic frequency output current, thereby causing said magnetostrictive ultrasonic transducer to vibrate at ultrasonic frequencies.

8. The apparatus of claim 7, wherein said magnetostrictive ultrasonic transducer vibrates at a vibrating frequency of between 28 and 32 kHz.

9. The apparatus of claim 7, wherein said magnetostrictive ultrasonic transducer vibrates at a vibrating frequency of 30 kHz.

10. The apparatus of claim 7, wherein said applicator blade has a reciprocating amplitude of vibration of between 12 and 37 microns.

11. The apparatus of claim 7, wherein said applicator blade has a reciprocating amplitude of vibration of 25 microns.

12. The apparatus of claim 5, wherein said magnetostrictive ultrasonic transducer is releasably securable to said handle and said coupler acts between said stack and said applicator.

13. The apparatus of claim 12, wherein said fluid conduit means includes at least one irrigation passage extending from said cavity and through said coupler.

14. The apparatus of claim 1, wherein said applicator is releasably securable to said coupler.

15. The apparatus of claim 1, wherein said applicator is constructed of titanium.

16. The apparatus of claim 15, wherein said titanium is 6AL4V titanium.

17. The apparatus of claim 15, wherein said titanium has a grain direction aligned with a longitudinal axis of said applicator.

18. The apparatus of claim 5, wherein said applicator includes a connector portion extending from said applicator blade, for securing said applicator to said coupler.

19. The apparatus of claim 18, wherein said fluid conduit means includes at least one irrigation passage extending through said connector portion.

20. The apparatus of claim 1, wherein a standing wave is associated with said reciprocating motion of said applicator, said standing wave having nodes, which correspond to areas of minimum reciprocating amplitude of displacement, and anti-nodes, which correspond to areas of maximum reciprocating amplitude of displacement, wherein said applicator is of a length such that said applicator blade is located at an anti-node.

21. The apparatus of claim 1, wherein said applicator blade includes at least one abrading surface.

22. The apparatus of claim 1, wherein said applicator blade includes two abrading surfaces.

23. The apparatus of claim 21, wherein said at least one abrading surface includes at least one ridge extending transversely across said at least one abrading surface and defining a ridge abrading surface.

24. The apparatus of claim 22, wherein said two abrading surfaces each include at least one ridge extending transversely across each abrading surface and defining respective ridge abrading surfaces.

25. The apparatus of claim 23, wherein said at least one ridge extends transversely across said at least one abrading surface, adjacent to an outer edge.

26. The apparatus of claim 1, wherein said coupler includes a shaft extending to said applicator blade, said shaft having a central axis, wherein said applicator blade is offset from said central axis to enable said apparatus to be operated with said handle above said plane of said surface being abraded.

27. The apparatus of claim 1, wherein said coupler includes a shaft extending to said applicator, said shaft having a central axis, wherein said applicator blade is in-line with said central axis.

28. The apparatus of claim 27, wherein said applicator blade is spatulate in shape.

29. The apparatus of claim 28, wherein said applicator blade includes a flared portion and a rectangular portion.

30. The apparatus of claim 29, wherein said rectangular portion is offset from said 5 central axis to enable said apparatus to be operated with said handle above said plane of said surface being abraded.

31. The apparatus of claim 30, wherein said rectangular portion meets said flared portion at an angle of between 90 degrees and 180 degrees relative to said central axis.

32. The apparatus of claim 31, wherein said angle is 150 degrees.

33. The apparatus of claim 31, wherein said angle defines an outer vertex, adjacent to which at least one ridge extends and defines a ridge-abrading surface.

34. The apparatus of claim 31, wherein said angle defines an outer vertex, on which at least one ridge extends and defines a ridge-abrading surface.

35. The apparatus of claim 1, wherein said applicator blade includes at least one hole extending therethrough for allowing fluid passage therethrough onto an underlying surface.

36. The apparatus of claim 34, wherein said at least one hole is a slot.

37. The apparatus of claim 35, wherein said at least one hole is located on said flared portion of said applicator blade.

38. The apparatus of claim 34 having a plurality of said holes.

39. An applicator, releasably securable to said handle of a skin abrading apparatus, including an applicator blade for applying a reciprocating abrading motion.

40. The applicator of claim 39, wherein said applicator is constructed of titanium.

41. The applicator of claim 40, wherein said titanium is 6AL4V titanium.

42. The applicator of claim 40, wherein said titanium has a grain direction aligned with a longitudinal axis of said applicator.

43. The applicator of claim 39, wherein said applicator includes a connector portion extending from said applicator blade, for securing said applicator to a coupler.

44. The applicator of claim 43, wherein said connector portion includes a fluid conduit means for directing fluid from a fluid supply onto said applicator blade.

45. The applicator of claim 44, wherein said fluid conduit means includes at least one irrigation passage for extending through said connector portion.

46. The applicator of claim 39, wherein a standing wave is associated with said reciprocating motion of said applicator blade, said standing wave having nodes, which correspond to areas of minimum reciprocating amplitude of displacement, and anti-nodes, which correspond to areas of maximum reciprocating amplitude of displacement, wherein said applicator is of a length such that said applicator blade is located at an anti-node.

47. The applicator of claim 39, wherein said applicator blade includes at least one abrading surface.

48. The applicator of claim 39, wherein said applicator blade includes two abrading surfaces.

49. The applicator of claim 47, wherein said at least one abrading surface includes at least one ridge extending transversely across said at least one abrading surface and defining a ridge abrading surface.

50. The applicator of claim 48, wherein said two abrading surfaces each include at least one ridge extending transversely across each abrading surface and defining respective ridge abrading surfaces.

51. The applicator of claim 49, wherein said at least one ridge extends transversely across said at least one abrading surface, adjacent to an outer edge.

52. The applicator of claim 39, wherein said applicator includes a connector portion extending from said applicator blade, said connector portion having a central axis, wherein said applicator blade is offset from said central axis to enable said apparatus to be operated with said shaft above said plane of said surface being abraded.

53. The applicator of claim 39, wherein said applicator includes a connector portion extending from said applicator blade, said connector portion having a central axis, wherein said applicator blade is in-line with said central axis.

54. The applicator of claim 53, wherein said applicator blade is spatulate in shape.

55. The applicator of claim 54, wherein said applicator blade includes a flared portion and a rectangular portion.

56. The applicator of claim 55, wherein said rectangular portion is offset from said central axis to enable said apparatus to be operated with said shaft above said plane of said surface being abraded.

57. The applicator of claim 56, wherein said rectangular portion meets said flared portion at an angle of between 90 degrees and 180 degrees relative to said central axis.

58. The applicator of claim 57, wherein said angle is 150 degrees.

59. The applicator of claim 57, wherein said angle defines an outer vertex, adjacent to which at least one ridge extends and defines a ridge-abrading surface.

60. The applicator of claim 57, wherein said angle defines an outer vertex, on which at least one ridge extends and defines a ridge-abrading surface.

61. The applicator of claim 39, wherein said applicator blade includes at least one hole extending therethrough for allowing fluid passage therethrough onto an underlying surface.

62. The applicator of claim 60, wherein said at least one hole is a slot.

63. The applicator of claim 61, wherein said at least one hole is located on said flared portion of said applicator blade.

64. The applicator of claim 60 having a plurality of said holes.
